(19) United States
(12) Patent Application Publication
(10) Pub. No.: US 2026/0294270 A1
(43) Pub. Date: Oct. 1, 2026

(54) APPARATUS AND METHODS FOR CAPTURING NON-VOLATILE AND SEMI-VOLATILE SUBSTANCES FROM BREATH

(71) Applicant: CANNABIX TECHNOLOGIES INC., Burnaby (CA)

(72) Inventors: Jared BOOCK, Gainesville, FL (US); Mikko MÄÄTTÄ, Burnaby (CA); Rajpaul ATTARIWALA, Burnaby (CA)

(21) Appl. No.: 19/118,714

(22) PCT Filed: Jul. 17, 2023
(86) PCT No.: PCT/CA2023/050957

§ 371 Date: Apr. 4, 2025

Related U.S. Application Data
(60) Provisional application No. 63/414,393, filed on Oct. 7, 2022.

(51) Int. Cl. A61B 5/08; A61B 5/00; A61B 5/097; A61B 10/00; G01N 1/40; G01N 1/42; G01N 33/497

(52) U.S. Cl. CPC A61B 5/082; A61B 5/097; A61B 5/4845; A61B 10/00; G01N 1/405; G01N 1/42; G01N 33/4975; A61B 2010/0087

(57) ABSTRACT

Methods and apparatus for capturing exhaled aerosols containing a target analyte of interest from breath, retaining the exhaled aerosols including the target analyte of interest in droplet form, and allowing the target analyte of interest to be desolvated directly from the droplet. The apparatus can use a filamentous substrate to capture the exhaled droplets.

### CROSS-REFERENCE TO RELATED APPLICATIONS

This application claims priority to, and the benefit of, U.S. provisional patent application No. 63/414393 filed 7 Oct. 2022, the entirety of which is incorporated by reference herein in its entirety.

### TECHNICAL FIELD

Some embodiments relate to apparatus or methods for capturing non-volatile and/or semi-volatile substances from a breath sample of a mammalian subject. Some embodiments relate to apparatus or methods for releasing the captured non-volatile and/or semi-volatile substances from a breath sample for further analysis. Some embodiments relate to apparatus or methods for capturing non-volatile and/or semi-volatile substances from a breath sample of a mammalian subject and providing the non-volatile and/or semi-volatile substances for further analysis. In some embodiments, the non-volatile and/or semi-volatile substances are lipophilic substances.

### BACKGROUND

### Breath Analysis

Many advancements have been made recently in the emerging field of human breath analysis, which offers many advantages over other methods for clinical and toxicology applications.

Human breath contains a combination of thousands of volatile organic compounds (VOCs), as well as aerosols which originate in the deep lung that carry particles such as proteins, lipids, even bacteria and viruses, as well as other substances, such as oils. All of these can reveal a great deal of information about the health and diet of the person and whether or not the person's body contains toxic chemicals from drugs, environmental contaminants, or pollutants. Changes in combinations of biomarkers can reveal the presence of certain diseases, or the efficacy of treatment.

Clinical analyses currently rely primarily on blood and urine, using the “gold standard” for analysis: mass spectrometry. All of these require sample preparation and significant time for the chromatography step. In addition, the diagnosis of several diseases is conducted by tissue biopsy, which is intrusive, expensive, and time-consuming, and, in many cases, is still not adequate for early detection.¹⁻¹⁵

Recent studies have shown the potential of breath analysis for the determination of the presence of several diseases, such as: respiratory infections, cancers (lung, breast, bladder, prostate), liver disease, kidney disease, diabetes, and bowel diseases. Other studies have shown the feasibility of employing breath testing for the detection of drugs in the body, including the possible use of breath as the standard of detection, rather than blood.¹⁶⁻²⁰

### Non-Volatile and Semi-Volatile Analytes in Human Breath

In the field of detecting the presence of alcohol (specifically ethanol) in the system of a person, it is common to use a breathalyzer, which is a hand-held device that can be used to estimate the blood alcohol content of a subject based on the amount of alcohol present in the subject's breath. Ethanol is a hydrophilic substance that is miscible in water, and further is expelled from the lungs in reasonable quantities, making breathalyzers reasonably reliable for estimating the blood alcohol content of a subject based on a breath sample. Breathalyzers therefore can easily be used by law enforcement officials to facilitate immediate roadside screening of drivers suspected to have a blood alcohol level that is above the legally permissible limit.

With the legalization of cannabis in many jurisdictions, there is a strong public interest in ensuring that drivers are not driving while their judgment is unduly impaired by the recent consumption of cannabis. However, there is currently no device that reliably captures and detects the presence of Δ⁶-tetrahydrocannabinol (THC) or other cannabinoids or other non-volatile or semi-volatile substances in breath. It is very difficult to collect consistent samples of such non-volatile or semi-volatile substances from breath. For example, ethanol is far more volatile than THC, having a vapor pressure on the order of about 59 mmHg at 25° C. compared to THC with a vapor pressure of 4.6×10⁻⁸ mmHg at 25° C. (2.57×10⁻⁵ Pa for Δ⁹-THC³³), making THC much more difficult to obtain in a breath sample.

Existing breath sampling methods rely on adsorption of the analyte of interest, followed by desorption by either heat or solvent extraction. THC and other cannabinoids, for instance, have unique chemical properties that result in extremely low collection efficiencies using these methods. One of these is that the vapor pressure at ambient conditions has been measured to be 2.57×10⁻⁵ Pa for Δ⁹-THC³³, which is extremely low, meaning that THC will not readily enter the gas phase. In addition, cannabinoids are a sticky resin; it has been shown that it is difficult to remove them from surfaces once contact is made.

One of the processes by which the lungs remove foreign particles is the generation of aerosols. For instance, following consumption, cannabinoids, which are non-miscible in water, become distributed as a two-phase liquid emulsion with the predominantly water-containing fluid that lines airway walls. A surfactant in the airway fluid aids in this process. When turbulent air passes over the epiglottis, this fluid is disturbed, forming an aerosol, which is expelled with breath exhalation, taking foreign particulate matter from the lungs with it. Nearly all THC and other cannabinoids remain in the liquid condensed from breath aerosol. Higher velocity in the airways, especially on inhalation, produces more aerosol, which removes more particles.²¹⁻²³ In this way, cannabinoids enter the breath from the blood during alveolar gas exchange in the lungs.

Detection of cannabinoids in breath may be a better indicator of impairment than measurements in saliva, blood or urine, as THC remains in breath for only a short period of time (e.g. 1-3 hours), which coincides with the period of peak impairment. In contrast, THC can remain in other bodily fluids for many hours, days, or even weeks after smoking (e.g. up to 24 hours for oral fluid, up to three weeks for blood, and up to one month for urine), which is beyond the period of impairment.³⁵

One of the main limitations of current breath collection methods is that breath aerosol droplets containing analyte particles are not transferred efficiently. They are separated from VOCs and lost, either by intentional removal via an interposing apparatus such as a mouthpiece, or when the droplets deposit on a surface within the collection system, and are allowed to desolvate, which leaves the analyte particle on the surface. In many cases, such as oils, it can be difficult to remove the analyte from the surface once it is adsorbed. This, then, results in poor sample collection efficiencies for semi-and non-volatile analytes.

### One-Dimensional Structures as a Capture and Collection Substrate for Breath Aerosol Droplets

In arid regions, efficient water droplet collection methods are needed and employed. One of these is the one-dimensional structure, such as spider webs, needles, and other filaments. Water droplets are collected on filaments with minimal contact angle, and it is possible for them to be transported along the filament. Water droplets can also be collected between two filaments, as a liquid bridge. The distance between filaments, thickness, and material can all be optimized to control the size of the droplets that are captured.²⁴⁻³²

There remains a need for a device and/or method to facilitate the convenient and non-invasive roadside identification of drivers who are under the influence of cannabis or other drugs. Examples of non-volatile or semi-volatile substances that cannot readily be detected in human breath but which it might be desirable to detect include nicotine, caffeine, fentanyl, and the like.

The foregoing examples of the related art and limitations related thereto are intended to be illustrative and not exclusive. Other limitations of the related art will become apparent to those of skill in the art upon a reading of the specification and a study of the drawings.

### SUMMARY

The following embodiments and aspects thereof are described and illustrated in conjunction with systems, tools and methods which are meant to be exemplary and illustrative, not limiting in scope. In various embodiments, one or more of the above-described problems have been reduced or eliminated, while other embodiments are directed to other improvements.

One aspect provides a method of analyzing for a presence of a target substance in breath of a subject. A breath sample is collected from the subject, aerosol droplets from the breath containing both water and the target substance are collected on a collection medium, and the aerosol droplets are desolvated from the collection medium for analysis. After the aerosol droplets have been collected, the collection medium can be stored under storage conditions for a storage period. The storage conditions can be refrigerated or freezing conditions, or room temperature. The storage period is a period of time that is shorter than a length of time required for the water in the aerosol droplets to fully evaporate under the storage conditions. After the storage period, the aerosol droplets can be desolvated from the collection medium, e.g. for further analysis. The collection medium can be a filamentous substrate such as silica wool, graphite fiber, or metal wool. The aerosol droplets can be coalesced during collection. Allowing the aerosol droplets to flow past a droplet diverter and/or through at least one knife-like ridge structure that has an aperture with an internal diameter that narrows in a downstream direction can be used to coalesce the aerosol droplets.

One aspect provides a method of collecting a target substance from breath of a subject. The breath sample is obtained from the subject, and aerosol droplets containing both water and the target substance are permitted to collect on a collection medium. The aerosol droplets are retained in liquid form on the collection medium.

One aspect provides an apparatus for collecting aerosol droplets containing a target substance from breath. The apparatus has a mouthpiece, a collection cartridge in fluid communication with the mouthpiece, and a filamentous substrate for collecting the aerosol droplets, the filamentous substrate being contained within the collection cartridge. The filamentous substrate can be silica wool, graphite fiber or metal wool. A puck can be provided to sealingly interpose the collection cartridge and the mouthpiece. The puck may have one or more knife-like ridges at a downstream end of the puck. The one or more knife-like ridges may have an internal aperture with a diameter that narrows in a downstream direction. The mouthpiece may have a droplet diverter positioned to promote coalescence of aerosol droplets from a breath sample.

In addition to the exemplary aspects and embodiments described above, further aspects and embodiments will become apparent by reference to the drawings and by study of the following detailed descriptions.

### BRIEF DESCRIPTION OF THE DRAWINGS

Exemplary embodiments are illustrated in referenced figures of the drawings. It is intended that the embodiments and figures disclosed herein are to be considered illustrative rather than restrictive.

FIG. 1 shows a sectional view of an example embodiment of an apparatus for capturing aerosol droplets containing one or more target analytes in the aerosol droplets.

FIG. 2 shows a sectional view of a second example embodiment of an apparatus for capturing aerosol droplets containing one or more target analytes in the aerosol droplets.

FIG. 3A shows a photograph of a mouthpiece illustrating how tapered airflow structures can assist in coalescing aerosol droplets into larger droplets.

FIG. 3B illustrates a cross-sectional view of an example embodiment of a puck illustrating the knife-like ridge structures formed at a downstream tip thereof.

FIG. 4 shows a photograph of coalesced aerosol droplets on an example embodiment of a filamentous substrate.

FIGS. 5A and 5B show photographs of example embodiments of filamentous substrates that have been formed into a net to capture coalesced aerosol droplets within a collection cartridge.

FIG. 6 illustrates an example embodiment showing a mouthpiece, a puck and a collection cartridge separated from one another.

FIG. 7 shows schematically an example embodiment in which two collection cartridges are deployed in parallel to collect aerosol droplets simultaneously from one single breath sample provided through a mouthpiece having a split in the fluid flow path.

FIG. 8 shows an example embodiment of a method for collecting particles of a target analyte present in aerosol droplets obtained from breath.

FIG. 9 shows an example embodiment of a method for evaluating particles of a target analyte present in aerosol droplets obtained from breath using mass spectrometry.

FIG. 10 shows a mass spectrum of a breath sample obtained using a cartridge containing a filamentous substrate according to one example embodiment.

### DESCRIPTION

Throughout the following description specific details are set forth in order to provide a more thorough understanding to persons skilled in the art. However, well known elements may not have been shown or described in detail to avoid unnecessarily obscuring the disclosure. Accordingly, the description and drawings are to be regarded in an illustrative, rather than a restrictive, sense.

As used herein, “upstream” refers to a direction towards a mouth of a user providing a breath sample. “Downstream” refers to a direction away from the mouth of the user providing a breath sample.

The inventors have now discovered that non-volatile or semi-volatile substances, including for example Δ⁹-tetrahydrocannabinol (THC) and related compounds, are found in breath samples in the form of an aerosol in liquid droplets in the breath sample. The inventors have developed methods and apparatus to capture such non-volatile or semi-volatile substances from a breath sample obtained from a mammalian subject. The inventors have further developed methods and apparatus to allow the non-volatile or semi-volatile substances captured from the breath sample to be provided for further study or analysis, e.g. released from the sampling apparatus for further analysis. In some embodiments, the non-volatile or semi-volatile substances are lipophilic substances.

More specifically, the inventors have developed a “two-stage” or “semi-direct” or “liquid secondary adsorption” method to sample for analytes of low volatility in breath. Such method involves agglomeration of aerosol droplets in exhaled breath by deposition and impact on walls and ridge structures and/or focusing of aerosol droplets from breaths via blade or needle-like structures and/or collection on a filamentous medium, partially mimicking the most efficient systems in nature for collection and transport of water droplets. Once collected and optionally held for a period of time, the droplets containing the analytes can be e.g. released, thermally desolvated, and passed for further analysis, e.g. passed into an ionizer, and ionized for downstream analysis, e.g. by mass spectrometry.

The inventors have demonstrated using experiments with cannabinoids that if desolvation is performed before the water in the captured droplets from exhaled breath is fully vaporized, then higher amounts of cannabinoids are collected; otherwise, they remain on surfaces of the collection device and are difficult to release from the collection device. The “two-stage” method offers the advantage of controlling the amount of sample that is collected, both for measurement standardization and preconcentration, as well as controlling the rate of droplet desolvation for optimal signal/noise. For example, excessive water in the ionizer can affect signal and produce unwanted cluster ions, so it may be desirable to control the size of droplets released upon droplet desolvation as described herein. In addition, with this method, samples can be either collected and analyzed immediately, or stored for additional analysis or confirmation/validation. Without being bound by theory, the filamentous medium may also increase pressure resistance during breath collection, which can result in delivery of more alveolar air from subjects, which can further increase the amount of analyte collected.

In the “two-stage” method, breath aerosol droplets serve as both a carrier and a secondary adsorbent. In this context, the target analyte molecules remain adsorbed in the water of the aerosol droplets, rather than being adsorbed on the surface of the collection device. Minimal contact is made with any solid surfaces, including the filamentous substrate, preventing analyte loss by adsorption onto surfaces. Droplets may be propelled primarily by aerodynamics. There are multiple possible ways to minimize contact between the aerosol droplets and solid surfaces. For example, the Leidenfrost Effect can be employed to prevent droplet-surface contact. The Leidenfrost Effect occurs when a liquid that is close to a surface that is significantly hotter than the liquid's boiling point produces an insulating vapor layer that keeps the liquid from boiling rapidly. Because of this repulsive force, a droplet hovers over the surface, rather than making physical contact with it. Alternatively or additionally, the filamentous substrate can be chemically inert and/or its surface properties can be optimized for minimal droplet contact. Efficient analyte transport, in turn, can lead to smaller overall breath sample sizes needed. This method differs from standard thermal desorption in that the breath aerosol itself is used as a carrier, minimizing sample loss throughout the system.

Thermal droplet desolvation may be done directly, indirectly, or a combination of both. The filamentous substrate can take different possible shapes or forms, and can be made of different materials with desirable surface properties, such as silica wool, graphite fiber, metal wool or the like. The filamentous substrate can be packed at different possible densities and/or in layers for optimal droplet collection; the filaments can consist of different possible diameters. The filamentous medium may also be thermally and/or electrically conductive.

Most aerosol droplets are microscopic, as small as 0.05 μm; though, they can be as large as 500 μm when expelled from the body at a high velocity.¹⁰ It is possible to focus and coagulate droplets into larger sizes, which increases the amount of analyte present. This can be used as a preconcentration method. By controlled heating, for example in and leading up to the ionization region in embodiments in which the analyte is analyzed by mass spectrometry, the analyte is desolvated prior to ionization. It is also possible to collect the analyte via solvent extraction.

### Mechanisms of the Two-Stage Method

Without being bound by theory, the “two-stage” method has two confirmed mechanisms that can be used to implement various embodiments of the method.
1. Optionally concentrate aerosol droplets on the inside surface of a collection tube (e.g. made from alumina ceramic) and then pass these droplets (e.g. having a diameter of ~1 mm) are from the collection tube (e.g. by being blown out) so they are caught on a filamentous medium, e.g. a thin-layer silica wool, graphite fiber, metal wool or other similar net lining the collection cartridge. In alternative embodiments, in addition to or rather than first using a collection tube, the aerosol droplets can be coalesced using a droplet diverter and/or a puck or other intermediate structure containing knife-like ridges having an aperture therethrough with a diameter that narrows in the downstream direction and then passed to the filamentous medium. Droplets make contact with the walls and any provided ridge structures and impact each other and coalesce, until the droplets reach a desired size. Further, the droplets can be focused onto the tip of a blade or needle-like structure, for example before being passed to the filamentous medium.
2. Direct droplet deposition on a filamentous substrate without prior concentration in a collection tube. The droplets can then be directly released from the filamentous substrate by thermal desolvation.

For example, as illustrated in FIG. 1, in an example embodiment of an apparatus 10 collecting aerosol droplets from breath, a mouthpiece 12 in fluid communication with a generally tubular collection tube portion 14 is used to collect aerosol droplets 16 from a breath sample. While in the illustrated embodiment, mouthpiece 12 is illustrated as being integrally formed with collection tube 14, in alternative embodiments mouthpiece 12 and collection tube 14 may be formed as separate components, and may be made from different materials. For example, mouthpiece 12 may be made of any desired material, such as for example plastic to be easily disposable, whereas collection tube 14 is generally made from a material that is selected to minimize the adsorption of the aerosol droplets on the surface of collection tube 14, for example alumina. In some embodiments, mouthpiece 12 may be a commercially available mouthpiece usable with a plurality of different breath collection devices.

In some embodiments, the collection tube 14 is made from a relatively inert material such as alumina ceramic, graphite or the like, or any other material that is suitably treated e.g. with a suitable coating so as to be relatively inert, and is used to initially collect aerosol droplets 16 from an exhaled breath sample. In some embodiments, the collected aerosol droplets 16 coalesce and enlarge within collection tube 14 as additional breaths are supplied. The collected aerosol droplets 16 can then be blown out from collection tube 14 or otherwise expelled from collection tube 14 in any suitable manner (e.g. via gravity drop, vibration, via pushing with a carrier gas or pulling using suction applied by a pump, electrostatic forces, explosive heating, or the like, or a combination thereof) and caught on a thin-layer of filamentous substrate 18, e.g. a silica wool net, graphite fiber, metal wool or the like lining a collection cartridge 20. Any suitable material can be used for filamentous substrate 18, e.g. silica wool, graphite fiber, metal wool, or other material that limits the amount of interaction between the collected aerosol droplets 16 and filamentous substrate 18 and does not chemically react with aerosol droplets 16 and the target analyte, e.g. silica, graphite, carbon, or the like. In some embodiments, collection cartridge 20 includes a coarse mesh outlet 22, so that air, for example from exhaled breath which moves in direction of flow 30 from the mouthpiece 12 to the coarse mesh outlet 22, can freely pass through and exit collection cartridge 20, while aerosol droplets 16 are captured as described above.

In some embodiments, collection tube 14 can be omitted, and the aerosol droplets 16 can be collected directly in filamentous substrate 18 from exhaled breath. In such embodiments, the aerosol droplets 16 may not coalesce or may coalesce to a lesser extent than in embodiments in which collection tube 14 is employed.

In some embodiments, a pump or other airflow apparatus may be used to apply a mild suction in the direction of flow 30 of the subject's breath. Without being bound by theory, it is believed that the use of a pump to help pull the breath sample through the filamentous substrate 18 may assist in capturing the aerosol droplets 16 on the filamentous substrate 18.

In some embodiments, the filamentous substrate 18 is packed into a collection cartridge 20. The cartridge can be any shape, size, or material, e.g. aluminum in some embodiments. A breath sample passes through the collection cartridge 20 and any breath aerosol is captured on the filamentous substrate 18. One example embodiment of a cartridge is an aluminum cylinder. Subsequent to sample collection, and optionally after a storage period, the collection cartridge 20 including the filamentous substrate 18 contained therein is inserted into a heater, the aerosol droplets 16 are released from the cartridge by a force such as carrier gas (e.g. nitrogen), explosive heating, vibration, or sonication, or the like, or a combination thereof. The released droplets 16 are heated and desolvated, separating the target analyte from the water in the aerosol droplets 16. In some embodiments, the filamentous substrate 18 is wound into a net-like structure, e.g. as described below as 140 and as illustrated in FIGS. 5A and 5B and the resultant net-like structure is nestled within collection cartridge.

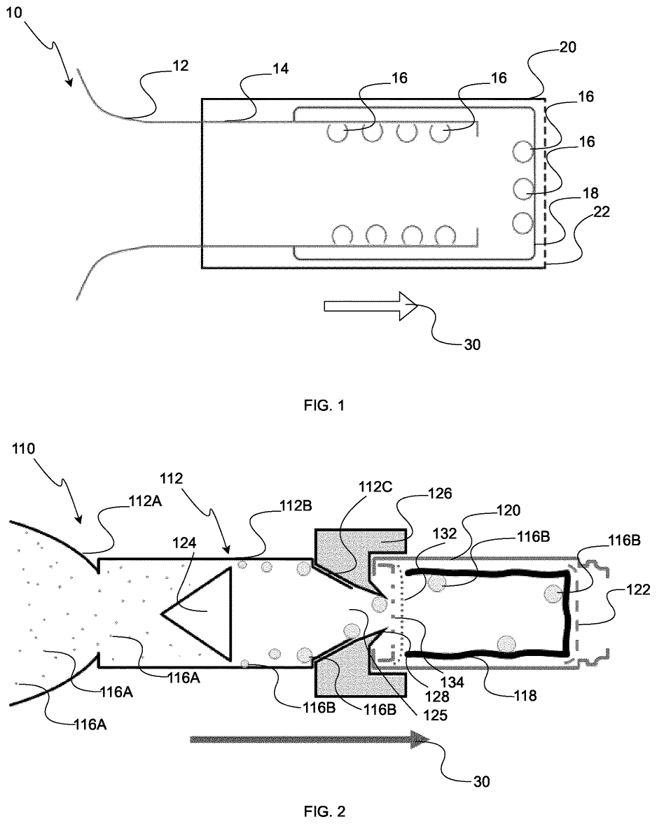

With reference to FIG. 2, a second example embodiment of an apparatus 110 for collecting aerosol droplets is illustrated. Collection apparatus 110 is generally similar to collection apparatus 10, and components of collection apparatus 110 that perform a similar function to components of collection apparatus 10 are illustrated with reference numerals incremented by 100 and any of the materials or apparatus previously described with reference to collection apparatus 10 can be used to manufacture corresponding components of collection apparatus 110. Any of the methods or other associated components described with reference to either of collection apparatus 10 or 110 can be applied to or used with collection apparatus 110 or 10, respectively.

Collection apparatus 110 has a mouthpiece 112 (shown as 112A, 112B and 112C as described further below) into which a user of the apparatus blows. In the illustrated embodiment, the mouthpiece has an upstream component 112A into which the user blows the breath sample, and a downstream component 112B, through which the collected breath flows towards collection cartridge 120.

Downstream component 112B of the mouthpiece 112 has a droplet diverter 124 which redirects the flow of fluid through downstream component 112B and encourages exhaled aerosol droplets 116A to contact one another and coalesce to form coalesced aerosol droplets 116B. As exhaled aerosol droplets 116A travel in the direction of flow 30, the droplets continue to coalesce with one another and with coalesced aerosol droplets 116B (which can further coalesce with other coalesced aerosol droplets 116B), so that the droplets continue to increase in diameter as they travel in the direction of flow 30. In some embodiments, mechanisms to prevent undesired oral fluids such as spittle from proceeding past mouthpiece 112 can be used, for example a spit-trap as may be commonly provided in various mouthpieces intended for use with various breath collection devices, or a thin piece of plastic film could be used to block spit, or so on.

In the illustrated embodiment, collection apparatus 110 is provided with an intermediate bridge between the mouthpiece 112 and collection cartridge 120, which is referred to as a puck 126. Puck 126 includes one or more claws or knife-like ridge structures 128 having an aperture 125 with a tapering internal diameter in the downstream direction, which further directs and focuses the flow of coalesced aerosol droplets 116B towards one another and encourages further coalescence of the aerosol droplets. In this way, the puck 126 acts as a pre-concentration stage for the exhaled breath, further concentrating the exhaled aerosol droplets 116A and the coalesced aerosol droplets 116B. In some embodiments, knife-like ridge structures 128 have a generally trapezoidal three-dimensional shape, and the interior diameter of aperture 125 of knife-like ridge structures 128, and in particular the downstream exit ends thereof, can be selected depending on the optimal size of coalesced aerosol droplets 116B that it is desired to have entering collection cartridge 120.

In the illustrated embodiment, downstream portion 112B of the mouthpiece 112 includes a tapered end 112C, which has a complementary shape to the interior aperture 125 of knife-like ridge 128 of puck 126 so that mouthpiece 112 can be sealingly engaged with puck 126. Other shapes could be used for end 112C and end 112C does not necessarily need to be complementary in shape to or enter into puck 126 or aperture 125 as illustrated, provided that a good seal can be formed with puck 126. Further puck 126 may be made of a material that is well suited to form a strong seal when engaged with another component in a compression fit and/or further of a material that exhibits limited interaction with or adsorption of coalesced aerosol droplets 116B, for example polytetrafluoroethylene (PTFE, e.g. as sold under the trade name Teflon®), plastic material, or the like, to ensure a good sealing engagement between mouthpiece 112 and puck 126. In some embodiments, puck 126 may be manufactured using additive manufacturing techniques such as 3D-printing. Unlike components of collection cartridge 120, puck 126 is not subjected to heating during desolvation of the collected coalesced aerosol droplets 116B, and so the material from which puck 126 is made does not necessarily need to be able to withstand high temperatures. Puck 126 may contain a plurality of knife-like ridges 128, and each one of the knife-like ridges 128 encourages the further coalescence of coalesced aerosol droplets 116B.

Puck 126 is also in sealing engagement with collection cartridge 120, and is positioned upstream of collection cartridge 120 so that the collected breath and coalesced aerosol droplets 116B continue to flow in direction of flow 30 from puck 126 into collection cartridge 120. That is, puck 126 interposes both collection cartridge 120 and mouthpiece 112 and is in sealing engagement with both of the foregoing components. All of the foregoing components are in fluid communication with one another so that a breath sample can travel from mouthpiece 112 into collection cartridge 120 through puck 126.

In the illustrated embodiment, at the upstream end of collection cartridge 120, a fine mesh 132 is provided so that the incoming breath sample, including the coalesced aerosol droplets 116B, flows through fine mesh 132. Mesh inlet bars 134 can be provided to support and retain fine mesh 132 in position, e.g. in embodiments in which fine mesh 132 is relatively flexible. Fine mesh 132 assists with capturing and further coalescing coalesced aerosol droplets 116B for capture within collection cartridge 120, and can act as a sieve to help standardize the size of the coalesced aerosol droplets 116B that are captured on filamentous substrate 118 and/or released from filamentous substrate 118 during desolvation as described below (i.e. because droplets that are too large cannot pass through fine mesh 132). Fine mesh 132 can also assist in retaining filamentous substrate 118 within collection cartridge 120 (including e.g. if relatively small portions of filamentous substrate 118 break off).

In some embodiments, fine mesh 132 is a silk-like stainless steel mesh, aluminum mesh, or other relatively inert metal or other material able to withstand high temperatures and some mechanical stress and be formed into a mesh shape. When the coalesced aerosol droplets 116B are to be heated to desolvate a target analyte of interest contained therein for analysis, fine mesh 132 can assist with analyte desolvation by limiting the size of droplet that is passed out of collection cartridge 120 (e.g. because large droplets cannot pass through fine mesh 132 and instead are either broken into smaller droplets or are further heated while in contact with fine mesh 132 and released only once they have become smaller droplets) and can also help to ensure any particulate debris (e.g. from filamentous substrate 118) does not enter an analytical instrument, for example mass spectrometer.

After passing through fine mesh 132, the exhaled breath sample including coalesced aerosol droplets 116B continues to flow in direction of flow 30, and encounters filamentous substrate 118. Coalesced aerosol droplets 116B are captured by filamentous substrate 118, while air continues to flow therethrough. Exiting air can freely pass through a coarse mesh outlet 122 that is provided at the downstream end of collection cartridge 120 to contain filamentous substrate 118 in position. In some embodiments, coarse mesh outlet 122 is made from a metal mesh, although coarse mesh 122 may be made from any material that can withstand high temperatures without off-gassing and which can retain its shape without additional support (or if additional structural support is needed, a further structure similar to mesh inlet bars 134 could be provided to support coarse mesh 122 in alternative embodiments).

Together, fine mesh 132 and coarse mesh 122 contain filamentous substrate 118 within collection cartridge 120 to facilitate the capture and containment of coalesced aerosol droplets 116B inside collection cartridge 120.

Any suitable material can be used for collection cartridge 120. In some embodiments, collection cartridge 120 is made from a material that can readily be heated, and/or which can be heated evenly, to facilitate desolvation of target analytes from coalesced aerosol droplets 116B for analysis, e.g. by mass spectrometry. In some embodiments, collection cartridge 120 is made from alumina.

In some embodiments, a pump or other airflow apparatus may be used to apply a mild suction in the direction of flow 30 of the subject's breath within collection apparatus 110. Without being bound by theory, it is believed that the use of a pump to help pull the breath sample through the filamentous substrate 118 may assist in capturing the coalesced aerosol droplets 116B on the filamentous substrate 118.

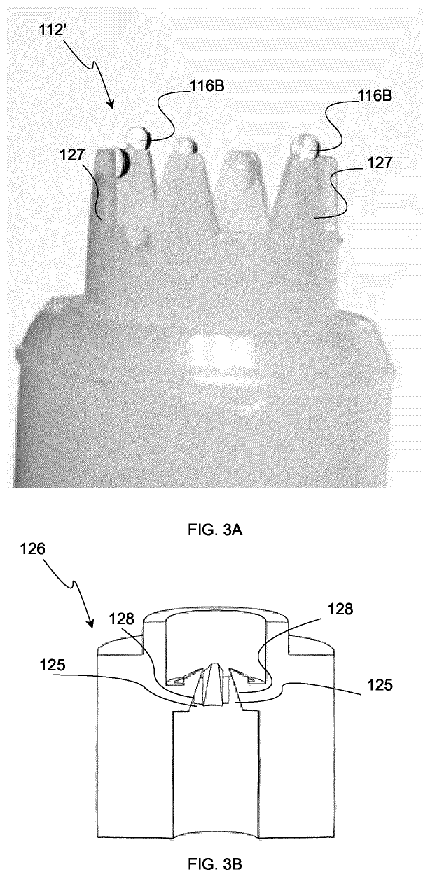

With reference to FIG. 3A, a photograph of an example embodiment of a mouthpiece 112′ that was tested to illustrate that tapered airflow structures 127 can assist in coalescing exhaled aerosol droplets 116A into larger coalesced aerosol droplets 116B is shown. FIG. 3A shows how tapered or trapezoidal structures having an internal diameter that narrows in the downstream direction can be used to coalesce exhaled aerosols into larger coalesced aerosol droplets 116B, and to focus those droplets onto the tip of the tapered airflow structures 127.

With reference to FIG. 3B, a cross-sectional view of an example embodiment of a puck 126 is shown. Knife-like ridge structures 128 protrude in the downstream direction from the body of puck 126 and form generally trapezoidal three-dimensional structures having an aperture 125 with an internal diameter that narrows in the downstream direction that act to focus and further promote the coalescence of coalesced aerosol droplets 116B. In some embodiments of the puck 126, the knife-like ridge structures 128 are omitted and puck 126 serves to seal the connection between the mouthpiece 112 and the collection cartridge 120, including providing sealing for any pump that may be used to assist in the collection of a breath sample using collection apparatus 110.

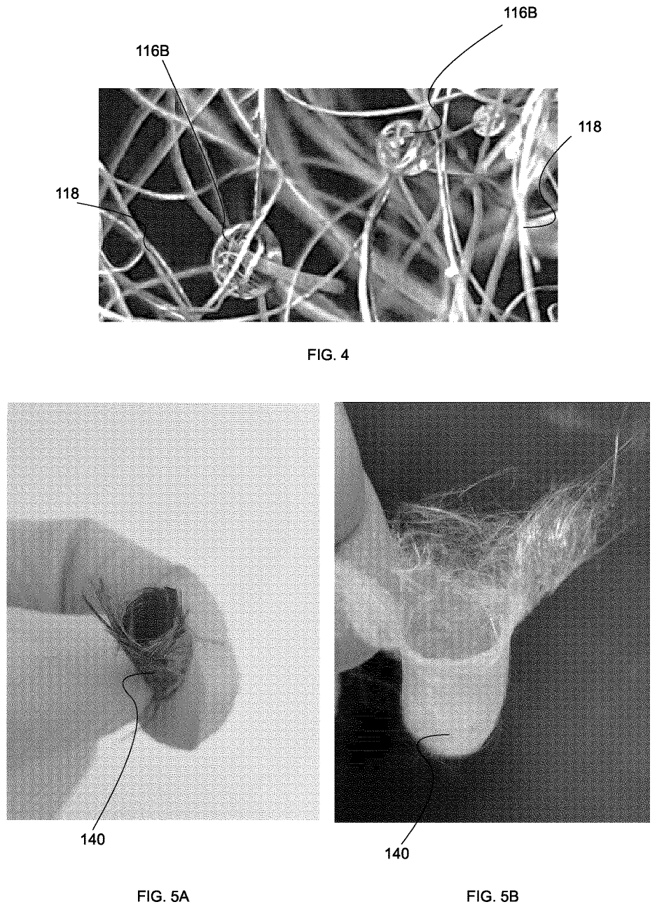

With reference to FIG. 4, after the coalesced aerosol droplets 116B exit puck 126 and enter collection cartridge 120, the coalesced aerosol droplets are captured and retained on filamentous substrate 118. As can be seen in FIG. 4, coalesced aerosol droplets 116B can remain in contact with the surface of filamentous substrate 118 with a minimum amount of contact. Thus, coalesced aerosol droplets 116B can be retained inside of collection cartridge 120, for example to facilitate their transfer to an analytical apparatus such as a mass spectrometer or their storage for later analysis. In some embodiments, after coalesced aerosol droplets 116B have been collected inside of collection cartridge 120, mouthpiece 112 is removed and optionally disposed of, and collection cartridge 120 can be either passed for immediate analysis, or sealed with a suitable cap and stored.

As best seen in FIGS. 5A and 5B, in some embodiments, the filamentous substrate 118 can be wound together and formed into a net 140 that is shaped and configured to sit inside of collection cartridge 120. For example, in embodiments in which collection cartridge 120 is cylindrically shaped, net 140 can be similarly cylindrically shaped. For example, net 140 can be formed by winding a filamentous substrate 118 around a cylindrical object such as a rod numerous times so that the multiple turns of the filamentous substrate 118 essentially weave together to form a self-supporting structure in the form of net 140. The density of the weave of the filamentous substrate 118 in net 140 can be selected to be sufficiently loose that air can still readily pass through the gaps between the filamentous substrate 118, but to be sufficiently tight that most or all of the coalesced aerosol droplets 116B will contact and therefore remain on filamentous substrate 118 rather than passing through net 140. In FIG. 5A, the pictured net 140 is formed from carbon fibres. In FIG. 5B, the pictured net 140 is formed from silica wool fibres.

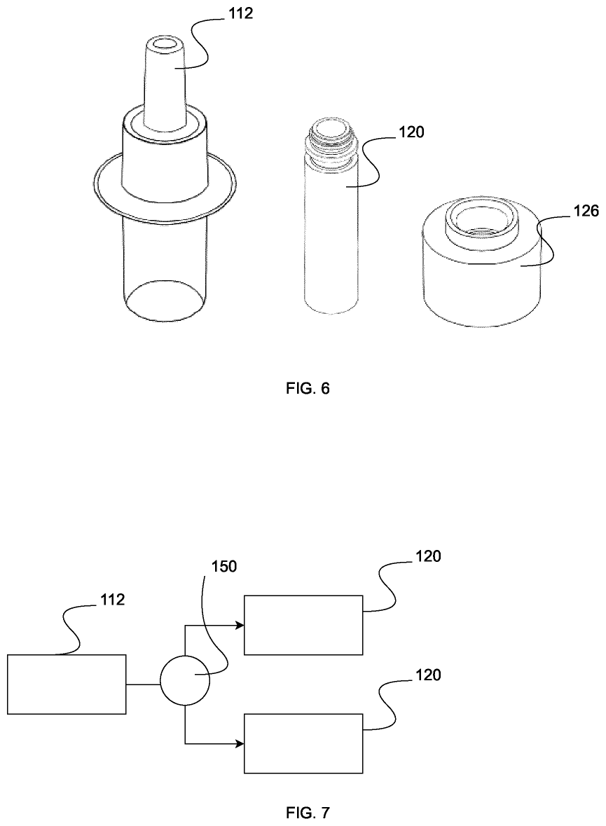

As best shown in FIG. 6, in some embodiments, mouthpiece 112, puck 126 and collection cartridge 120 are all detachably engageable from one another, so that these components can be manufactured from different materials. In some embodiments, mouthpiece 112 is disposable. In some embodiments, puck 126 and collection cartridge 120 are reusable, but are cleaned and sterilized between each use.

In some embodiments, as shown schematically in FIG. 7, two or more collection apparatus 10 or 110 can be deployed in parallel, so that a single breath sample is received in two or more collection apparatus 10 or 110. For example, the mouthpiece 112 used may include one or more splits 150 in the fluid flow path of the collected breath (e.g. in the form of a Y-shaped split or simply by providing two divergent fluid flow paths directly to a pair of side-by-side collection cartridges 20 or 120 from a single mouthpiece 112), so that one single breath sample provided to the mouthpiece can be supplied to two or more collection apparatus 10 or 110. In this way, for example, a first one of the collected samples can be immediately subjected to analysis, e.g. in a roadside setting, while a second one of the collected samples can be stored for subsequent analysis, e.g. in a laboratory setting, where more accurate instruments may be available to analyze the second one of the collected samples.

### Methods

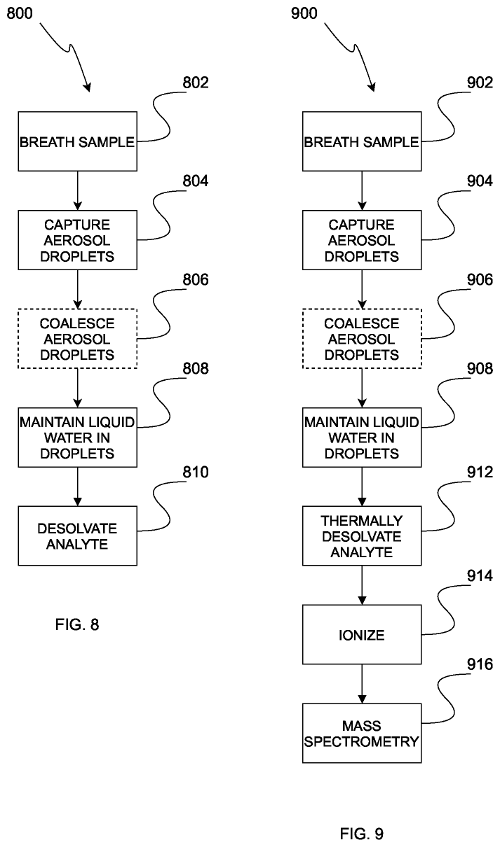

In some embodiments as illustrated with reference to FIG. 8, a method 800 of evaluating a target analyte present in aerosol droplets obtained from breath is provided. At 802, a breath sample is collected from a mammalian subject, which may be a human. The breath sample may be collected in any suitable manner, for example using a mouthpiece or the like.

At 804, aerosol droplets in the breath sample are captured. In some embodiments, an apparatus such as apparatus 10 or 110 described in this specification is used to capture the aerosol droplets. In some embodiments, the aerosol droplets are captured on a filamentous substrate. At 806, which may be conducted before or after step 808 described below and/or before or after the aerosol droplets are captured at step 804, the aerosol droplets are optionally coalesced together to form larger liquid drops containing the target analyte.

At 808 and throughout the duration of the time for which the aerosol droplets are held prior to desolvation for analysis at 810 (also referred to as a storage period), at least a portion of the water contained in the aerosol droplets is maintained in a liquid state. Maintaining at least a portion of the water contained in the aerosol droplets in a liquid state (i.e. not allowing water in the captured aerosol droplets to fully vaporize) assists in preventing deposition of the analytes entrained within the aerosol droplets on the collection apparatus, particularly non-volatile or semi-volatile substances which may have a tendency to remain on the surface of the collection apparatus if the aerosol droplets are permitted to reach a fully vaporized (i.e. dry) state within the collection apparatus.

In some embodiments, the storage period is between about 1 minute and about 50 hours, including any duration or subrange therebetween, e.g. about 2, 5, 10, 20, 30, 45, or 60 minutes, or about 1.5, 2, 5, 10, 15, 20, 24, 25, 30, 35, 40, 45 or 48 hours. In some embodiments, the sample can be refrigerated and/or frozen during the storage period. In some embodiments, if the sample is refrigerated or frozen, it can be stored for several days. In some embodiments, refrigeration involves storing the sample at a temperature between about 2° C. and about 15° C., including any value or subrange therebetween, e.g. 3, 4, 5, 6, 7, 8, 9, 10, 11, 12, 13 or 14° C. In some embodiments, freezing involves storing the sample at a temperature of less than about 0° C., e.g. between about −40° C. and about −2° C., including any value or subrange therebetween, e.g. −35, −30, −25, −20, −15, −10 or −5° C.

At 810, the target analyte is desolvated for analysis in any suitable manner, for example using any of the methods described in this specification, for example by thermally desolvating the collected aerosol droplets (so that the desolvated aerosol droplets can be analyzed in any method as desired, for example by passing the desolvated analyte to a mass spectrometer for analysis).

In some embodiments, during the conduct of method 800, the contact between the aerosol droplets and the apparatus used to capture the aerosol droplets from the breath sample is minimized in any suitable manner. For example, in some embodiments, the material from which the apparatus is made is chemically inert, e.g. the alumina ceramic material from which collection tube 14 or collection cartridge 20 or 120 is made in some embodiments and/or the material that is used for filamentous substrate 18 or 118 in some embodiments, and/or the material from which the apparatus is made is treated to modify its surface properties so as to minimize the contact between the material and aqueous water droplets, e.g. suitable hydrophobic coatings, suitable combinations of hydrophilic and hydrophobic coatings, surface texturing or the like. In some embodiments, the apparatus is heated so that the Leidenfrost Effect occurs, so that contact between the aerosol droplets and the material from which the apparatus is manufactured is minimized by the formation of a layer of steam between the material of the apparatus and the aerosol droplets.

In some embodiments, as illustrated with reference to FIG. 9, evaluation of the target analyte is carried out using mass spectrometry. For example, with reference to method 900, as for method 800, the breath sample is collected at 902, aerosol droplets are captured at 904 and optionally coalesced either before or after capture at 906, and at least a portion of the water in the aerosol droplets is maintained in a liquid state at 908, optionally for a storage period. At 912, the aerosol droplets including the analytes entrained therein are thermally desolvated, and then the analytes including the target analyte are ionized at 914 so that the target analyte can be analyzed by mass spectrometry at 916.

In some embodiments, at step 912, thermal desolvation is carried out in any suitable manner. For example, in some embodiments, the collection cartridge 20 or 120 containing the filamentous substrate 18 or 118 with the aerosol droplets 16 or coalesced aerosol droplets 116B is heated to a temperature in the range of about 120° C. to about 300° C., including any value or subrange therebetween, e.g. 125, 130, 140, 150, 160, 170, 180, 190, 200, 210, 220, 230, 240, 250, 260, 270, 280 or 290° C. In some embodiments, a multi-stage heating process is carried out. In some embodiments, heating may be carried out at a relatively lower temperature but for a relatively longer period of time than heating carried out at higher temperatures. In some embodiments, different heating methods could be used, e.g. conductive heating, flash heating, or the like.

In some embodiments, at steps 804 and 904, aerosol droplets are captured from breath using apparatus 10 or 110. In some such embodiments, collection tube 14 is present as part of apparatus 10. In other such embodiments, collection tube 14 is omitted as part of apparatus 10, and exhaled aerosol droplets 16 are collected directly on filamentous substrate 18.

In some embodiments, the component of breath to be detected, referred to herein also as a target substance or target analyte, is non-volatile or semi-volatile. In some embodiments, the component of breath to be detected is lipophilic. In some embodiments, the component of breath to be detected is fat-soluble. In some embodiments, the component of breath to be detected is a non-volatile molecule. In some embodiments, the component of breath to be detected is a semi-volatile molecule. In some embodiments, the component of breath to be detected is a hydrophobic molecule. In some embodiments, the component of breath to be detected is a cannabinoid, nicotine, caffeine, fentanyl, or the like. In some embodiments, the component of breath to be detected is 49-tetrahydrocannabinol (THC).

### EXAMPLES

Specific embodiments are further described with reference to the following examples, which are illustrative and not limiting in nature.

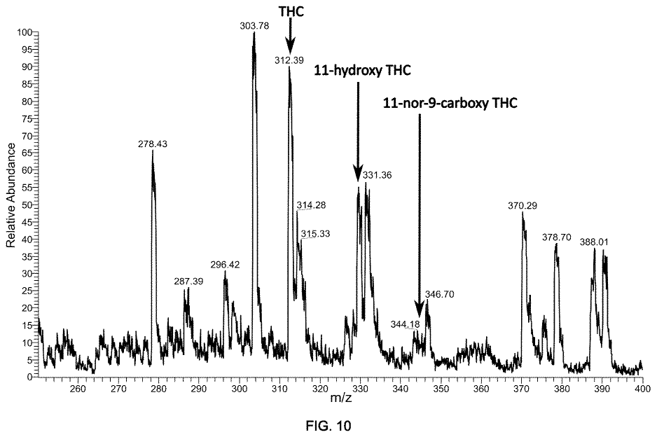

FIG. 10 shows a mass spectrum of a breath sample collected on a cartridge 15 minutes after a subject consumed cannabis. Not only is THC clearly visible, but also two of its metabolites, 11-hydroxy THC and 11-nor-9 carboxy THC are visible as labelled. Cannabinoids all exhibit low volatility, and are difficult to collect in breath samples. These metabolites are often considered “nondetectable” by standard thermal desorption methods.

The sample consists of five breaths captured using the “two-stage” method on a sample cartridge. A nitrogen carrier gas was used to release the collected aerosol from the cartridge, and the sample was heated to 175° C. to desolvate the target molecules prior to conducting mass spectrometry.

In another example, the inventors have obtained preliminary results that show that THC obtained and stored using apparatus such as apparatus 10 or 110 as described herein can be stored for a storage period prior to analysis. In preliminary lab experiments using THC tincture, an approximately 40% drop in areas under the curve (AUCs) were recorded during the first 2 days after sample collection when cartridges were stored in a freezer. After one week AUCs were about 30% of the original levels. If cartridges were stored at room temperature, the recorded drop in AUCs during the first 2 days was almost 70% after which the levels plateaued. In both cases the fragment peaks were still clearly visible above the noise level. Thus, the samples collected using the apparatus and methods as described herein can be stored for later analysis.

While a number of exemplary aspects and embodiments have been discussed above, those of skill in the art will recognize certain modifications, permutations, additions and sub-combinations thereof. It is therefore intended that the following appended claims and claims hereafter introduced are interpreted to include all such modifications, permutations, additions and sub-combinations as are consistent with the broadest interpretation of the specification as a whole.

### References

The following references are of interest with respect to the subject matter described herein. Each of these references is incorporated herein by reference in its entirety.
1. Cikach, F., et. al., Cardiovascular biomarkers in exhaled breath, Prog Cardiovasc Dis., 55(1): 34-43, 2014.
2. Schwarz, K., et. al., Characterization of exhaled particles from the healthy human lung—a systematic analysis in relation to pulmonary function variables, J. Aerosol Medicine and Pulmonary Drug Delivery, 23(6), 2010.
3. Gupta., J., et. al., Characterizing exhaled airflow from breathing and talking, Indoor Air, 20, 2010.
4. Mutlu, G., et. al., Collection and analysis of exhaled breath condensate in humans, Am J Respir Crit Care Med, 164, 2001.
5. Scheideler, L., et. al., Detection of nonvolatile macromolecules in breath: a possible diagnostic tool?, Am Rev Resp Dis, 148(3), 1993.
6. Drossinos, Y., et. al., Droplets and aerosols: an artificial dichotomy in respiratory virus transmission, Health Sci Rep., 4(275), 2021.
7. Horvath, I., et. al., A European Respiratory Society technical standard: exhaled biomarkers in lung disease, Eur Respir J, 49, 2016.
8. Schmidt, A., et. al., Portable exhaled breath condensate metabolomics for daily monitoring of adolescent asthma, J Breath Res, 14(2), 2020.
9. Bake, B., Exhaled particles and small airways, Resp Res, 20(8), 2019.
10. Larsson, P., et. al., Exhaled particles as markers of small airway inflammation in subjects with asthma, Clin Phys Func Imaging, 37, 2015.
11. Viklund, E., et. al., Severe acute respiratory syndrome coronavirus 2 can be detected in exhaled aerosol sampled during a few minutes of breathing or coughing, Influenza and other Respiratory Viruses, 16(402), 2022.
12. Brand, P., et. al., Inhaling to mitigate exhaled bioaerosols, Proceedings of the National Academy of Sciences, 101(50), 2004.
13. Fabian, P., Origin of exhaled breath particles from healthy and human rhinovirus infected subjects, J Aero Med and Pulm Drug Delivery, 24(3), 2011.
14. Bourouiba, L., Turbulent gas clouds and respiratory pathogen emissions: potential implications for reducing transmission of COVID-19, Clin Rev and Ed, 323(18), 2020.
15. J van Aerde, K., et. al., Non-invasive diagnostics of pathogenic bacteria using a breath sampler in children with cystic fibrosis, J Breath Res, 16, 2022.
16. Oluwasola, L., et. al., Exhaled breath analysis: a review of ‘breath-taking’ methods for off-line analysis, Metabolomics, 13(10): 110, 2017.
17. Das, S., et. al., Review-Non-invasive monitoring of human health by exhaled breath analysis: a comprehensive review, J. Electrochemical Society, 167(3), 2020.
18. Vereb, H., et. al., The possibilities will take your breath away: Breath analysis for assessing environmental exposure, Environmental Sci. Tech., 45(19), 2011.
19. Badjagbo, K., Potential of breath analysis: From environmental exposure assessment to medical diagnosis, Environmental Medicine, 3(3), 2012.
20. Beck, O., et. al., Detection of drugs of abuse in exhaled breath from users following recovery from intoxication, J. Analytical Toxicology, 36(9), 2012.
21. Asadi, S., et. al., Aerosol emission and superemission during human speech increase with voice loudness, Nat Sci Rep, 9(2348), 2019.
22. Almstrand, A., et. al., Airway monitoring by collection and mass spectrometric analysis of exhaled particles, Anal Chem, 81, 2009.
23. Johnson, G., et. al., The mechanism of breath aerosol formation, J Aero Med and Pulmonary Drug Delivery, 22(3), 2009.
24. Zhang, F., et. al., Bioinspired materials for water harvesting: focusing on microstructure designs and the improvement of sustainability, Mater Adv, 1(2592), 2020.
25. Zhu, H., et. al., Biomimetic water collecting materials inspired by nature, Chem Commun, 52(3863), 2016.
26. Romer, L., et. al., The elaborate structure of spider silk: structure and function of a natural high performance fiber, Prion, 2(4), 2008.
27. Pan Z., et. al., The upside down water collection system of Syntrichia caninervis, Nature Plants, 2(7), 2016.
28. Bedarkar, A., et. al., Capillary torque in a liquid bridge between two angled filaments, J App Phys, 106, 2009.
29. Wu, X., et. al., Droplets wetting on filament rails: surface energy and morphology transition, J Colloid and Interface Sci, 341, 2010.
30. Bedarkar, et. al., Wetting of droplets on two parallel filaments, App Surface Sci, 256, 2010.
31. Ju, J., et. al., Bioinspired One-dimensional materials for directional liquid transport, Acc Chem Res, 47, 2014.
32. Kim, Y., et. al., Topological heterogeneity and evaporation dynamics of irregular water droplets, Nat Sci Rep, 11(18700), 2021.
33. Lovestead, T., et. al., Determination of Cannabinoid Vapor Pressures to Aid in Vapor Phase Detection of Intoxication, Forensic Chem., 5: 79-85, 2017.
34. Walker, J., et. al., Accurate Representations of the Microphysical Processes Occurring during the Transport of Exhaled Aerosols and Droplets, ACS Central Science, 7:200-209, 2021.
35. Huestis, M.A. et. al., Bloodcannabinoids, Absorption of THC and formation of 11-OH-THC and THC-COOH during and after smoking marijuana, J. Anal. Toxicology, 16, p.276-282, 1992.

1. A method of analyzing for a presence of a target substance in breath of a subject, the method comprising the steps of:
collecting a breath sample from the subject;
allowing aerosol droplets containing both water and the target substance to collect on a collection medium;
prior to or during the step of collecting the aerosol droplets, coalescing the aerosol droplets; and
desolvating the aerosol droplets from the collection medium for further analysis, wherein the target substance comprises non-volatile or semi-volatile molecules.

2. The method as defined in claim 1, comprising, after collecting the aerosol droplets, storing the collection medium under storage conditions for a storage period, the storage period being a period of time that is shorter than a length of time required for the water in the aerosol droplets to fully evaporate under the storage conditions, and, after the storage period, conducting the step of desolvating the aerosol droplets from the collection medium.

3. The method as defined in claim 1, wherein the step of desolvating the aerosol droplets from the collection medium comprises thermal desolvation.

4. (canceled)

5. A method of collecting a target substance from a breath of a subject, the method comprising the steps of:
collecting a breath sample from the subject;
allowing aerosol droplets containing both water and the target substance to collect on a collection medium; and
retaining the aerosol droplets in a liquid form.

6. The method as defined in claim 1, wherein the collection medium comprises a filamentous substrate, or wherein the collection medium comprises silica wool, graphite fiber, or metal wool.

7. (canceled)

8. (canceled)

9. The method as defined in claim 1, wherein the step of coalescing the aerosol droplets comprises using a droplet diverter.

10. The method as defined in claim 1, wherein the step of coalescing the aerosol droplets comprises allowing the breath sample to flow through at least one knife-like ridge structure that has an aperture with an internal diameter that narrows in a downstream direction.

11. The method as defined in claim 1, wherein the aerosol droplets are collected on a collection tube prior to transferring the aerosol droplets to the collection medium.

12. The method as defined in claim 1, wherein a pump is used to pull the breath sample through the collection medium.

13. (canceled)

14. The method as defined in claim 2, wherein the storage conditions comprise ambient conditions, refrigerated conditions, or freezing conditions.

15. (canceled)

16. An apparatus for collecting aerosol droplets containing a target substance from breath, the apparatus comprising:
a mouthpiece;
a collection cartridge in fluid communication with the mouthpiece; and
a filamentous substrate for collecting the aerosol droplets, the filamentous substrate being contained within the collection cartridge; and
a puck sealingly interposing the collection cartridge and the mouthpiece, the puck comprising one or more knife-like ridges at a downstream end of the puck;
wherein the target substance comprises non-volatile or semi-volatile molecules.

17. The apparatus as defined in claim 16, comprising a collection tube in fluid communication with the mouthpiece, the collection tube interposing the mouthpiece and the filamentous substrate.

18. The apparatus as defined in claim 17, wherein the collection tube or the collection cartridge comprises alumina ceramic or aluminum, or wherein the filamentous substrate comprises silica wool, graphite fiber, or metal wool.

19. (canceled)

20. (canceled)

21. (canceled)

22. (canceled)

23. (canceled)

24. (canceled)

25. The apparatus as defined in claim 16, wherein the one or more knife-like ridges each comprise an internal aperture with a diameter that narrows in a downstream direction.

26. (canceled)

27. An apparatus for collecting aerosol droplets containing a target substance from breath, the apparatus comprising:
a mouthpiece;
a collection cartridge in fluid communication with the mouthpiece;
a filamentous substrate for collecting the aerosol droplets, the filamentous substrate being contained within the collection cartridge; and
a droplet diverter positioned to promote coalescence of aerosol droplets from a breath sample;
wherein the target substance comprises non-volatile or semi-volatile molecules.

28. The apparatus as defined in claim 16, further comprising a pump positioned to pull the breath downstream through the filamentous substrate.

29. The method as defined in claim 1, wherein the target substance comprises non-volatile molecules, hydrophobic molecules, lipophilic molecules, a cannabinoid, nicotine, caffeine, or fentanyl.

30. (canceled)

31. (canceled)

32. (canceled)

33. (canceled)

34. The apparatus as defined in claim 27, comprising a puck sealingly interposing the collection cartridge and the mouthpiece, wherein the puck comprises one or more knife-like ridges at a downstream end of the puck.

35. The method as defined in claim 1, wherein the step of collecting a breath sample from the subject comprises blocking oral fluids from proceeding past a mouthpiece used to collect the breath sample.

36. The apparatus as defined in claim 16, wherein the mouthpiece comprises a spit-trap to prevent oral fluids from proceeding past the mouthpiece.
